(19) United States
(12) Patent Application Publication
(10) Pub. No.: US 2025/0107799 A1
(43) Pub. Date: Apr. 3, 2025

(54) TENSIONABLE KNOTLESS TISSUE REPAIRS

(71) Applicant: ARTHREX, INC., Naples, FL (US)

(72) Inventors: Andrew Christian PETRY, Naples, FL (US); Peter J. DREYFUSS, Naples, FL (US); Christopher R. ADAMS, Naples, FL (US)

(21) Appl. No.: 18/420,828

(22) Filed: Jan. 24, 2024

Related U.S. Application Data
(60) Provisional application No. 63/586,076, filed on Sep. 28, 2023.

(51) Int. Cl. A61B 17/04

(52) U.S. Cl. CPC A61B 17/0487; A61B 2017/0404; A61B 2017/0445; A61B 2017/0456; A61B 2017/0462

(57) ABSTRACT

Systems and methods are provided for performing tensionable knotless tissue repairs. A locking ferrule that includes a one-way locking mechanism may be utilized as part of the tensionable knotless tissue repairs for tensioning and locking one or more strands of suture. The one-way locking mechanism may be established by one or more locking barbs of the locking ferrule.

### CROSS-REFERENCE TO RELATED APPLICATIONS

This application claims the benefit of U.S. Provisional Application No. 63/586,076, which was filed on Sep. 28, 2023 and is incorporated herein by reference in its entirety.

### BACKGROUND

This disclosure relates to the field of surgery, and more particularly to a variety of tensionable knotless tissue repairs.

Repetitive trauma to a joint, such as a knee, ankle, hip, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless tissue repairs. A locking ferrule that includes a one-way locking mechanism may be utilized as part of the tensionable knotless tissue repairs for tensioning and locking one or more strands of suture.

An exemplary locking ferrule for performing tissue repairs may include, inter alia, a body including an outer diameter wall, an inner diameter wall, and a cannulation that is circumscribed by the inner diameter wall. A plurality of locking barbs extend into the cannulation and are configured to lock a suture that is received through the cannulation relative to the body.

An exemplary surgical method may include, inter alia, loading a suture through a cannulation of a locking ferrule, tensioning the suture in a first direction, and locking the suture within the cannulation to prevent movement of the suture in a second direction.

Another exemplary surgical method may include, inter alia, loading a suture through a cannulation of a locking ferrule, inserting the locking ferrule into a socket formed in a bone, tensioning the suture in a first direction relative to the bone, and locking the suture within the cannulation to prevent movement of the suture in a second direction relative to the bone.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a locking ferrule that can be used for performing various tensionable knotless tissue repairs.

FIG. 2 is an end view of the locking ferrule of FIG. 1.

FIG. 3 is a cross-sectional view through section 3-3 of FIG. 1.

FIG. 4 is a cross-sectional view through section 4-4 of FIG. 2.

FIG. 5 schematically illustrates loading a suture through a locking ferrule.

FIG. 6 schematically illustrates locking a suture relative to a locking ferrule.

FIGS. 7, 8, 9, 10, 11, 12, 13, 14, 15, and 16 schematically illustrate a surgical method for performing a tissue repair.

FIGS. 17, 18, and 19 schematically illustrate another exemplary surgical method for performing a tissue repair.

FIG. 20 illustrates a suture locking system.

FIGS. 21A, 21B, 21C, and 21D illustrate another exemplary suture locking system.

### DETAILED DESCRIPTION

This disclosure relates to systems and methods for performing tensionable knotless tissue repairs. A locking ferrule that includes a one-way locking mechanism may be utilized as part of the tensionable knotless tissue repairs for tensioning and locking one or more strands of suture. These and other features of this disclosure are described in further detail below.

An exemplary locking ferrule for performing tissue repairs may include, inter alia, a body including an outer diameter wall, an inner diameter wall, and a cannulation that is circumscribed by the inner diameter wall. A plurality of locking barbs extend into the cannulation and are configured to lock a suture that is received through the cannulation relative to the body.

In any further embodiment, the plurality of locking barbs are integral features of the body.

In any further embodiment, each of the plurality of locking barbs includes a pointed tip.

In any further embodiment, the body extends along a longitudinal axis between a proximal end and a distal end.

In any further embodiment, each of the plurality of locking barbs is angled in a direction toward the proximal end.

In any further embodiment, the plurality of locking barbs each protrude inwardly from the inner diameter wall.

In any further embodiment, the plurality of locking barbs are arranged in at least a first row and a second row.

In any further embodiment, a first portion of the plurality of locking barbs of the first row are staggered relative to a second portion of the plurality of locking barbs of the second row.

In any further embodiment, the plurality of locking barbs are arranged across an entire length of the body.

In any further embodiment, the suture includes a varying thickness.

An exemplary surgical method may include, inter alia, loading a suture through a cannulation of a locking ferrule, tensioning the suture in a first direction, and locking the suture within the cannulation to prevent movement of the suture in a second direction.

In any further embodiment, a locking barb of the locking ferrule locks the suture relative to a body of the locking ferrule.

In any further embodiment, the locking barb protrudes inwardly from an inner diameter wall of the locking ferrule.

In any further embodiment, the locking barb engages a thickened section of the suture to prevent its movement in the second direction.

In any further embodiment, loading the suture through the cannulation includes pulling the suture through the cannulation in the first direction with a suture loader.

In any further embodiment, the method includes moving an eyelet of the suture loader in the second direction through the cannulation prior to pulling the suture through the cannulation.

In any further embodiment, pulling the suture through the cannulation includes pulling a thinned section of the suture through the cannulation.

Another exemplary surgical method may include, inter alia, loading a suture through a cannulation of a locking ferrule, inserting the locking ferrule into a socket formed in a bone, tensioning the suture in a first direction relative to the bone, and locking the suture within the cannulation to prevent movement of the suture in a second direction relative to the bone.

In any further embodiment, the locking ferrule is received over top of a fixation device that is positioned within the socket.

In any further embodiment, the fixation device is a suture anchor or a suture button.

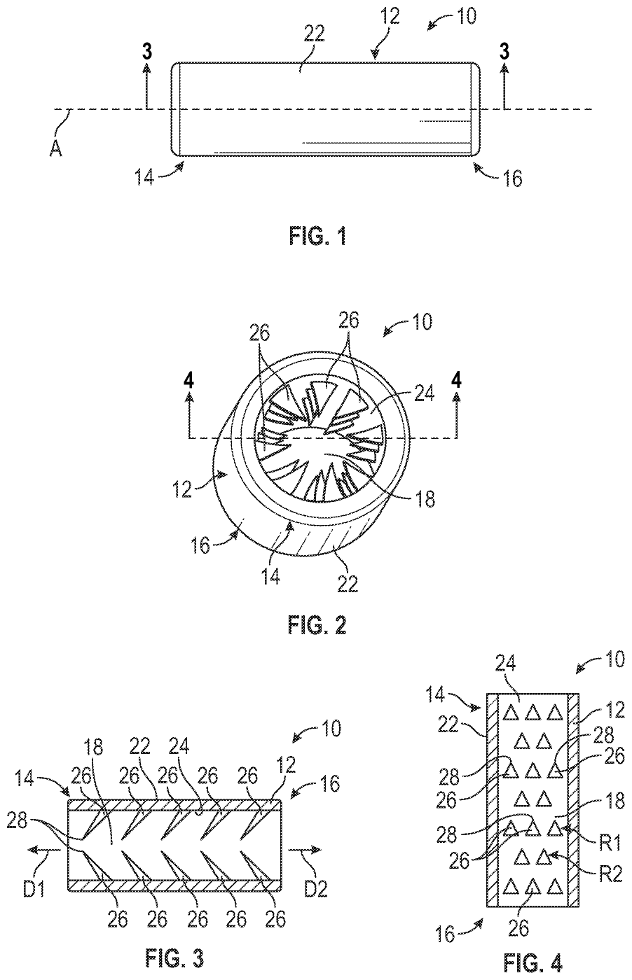

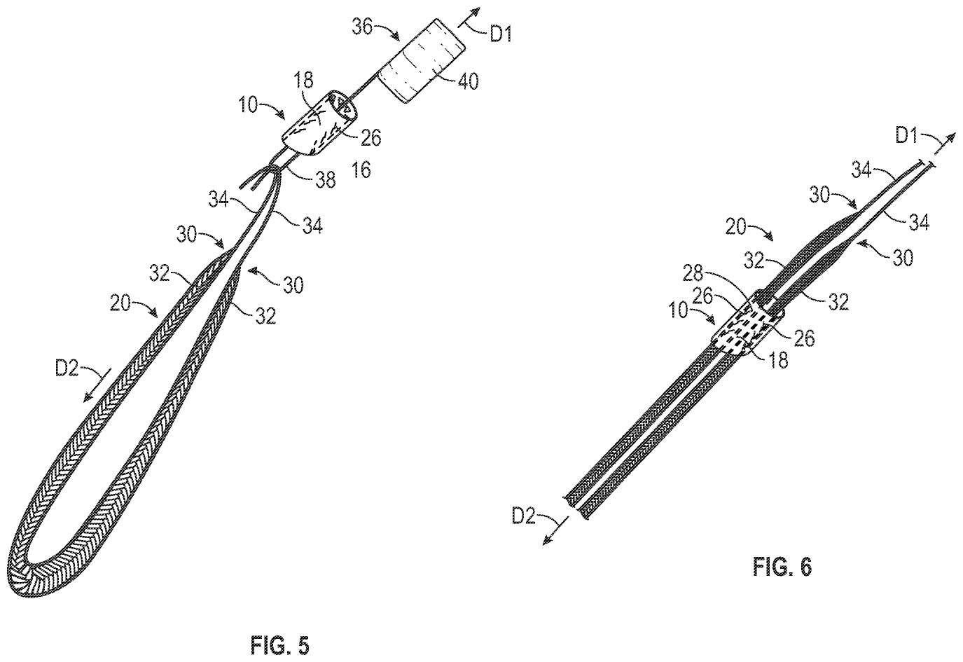

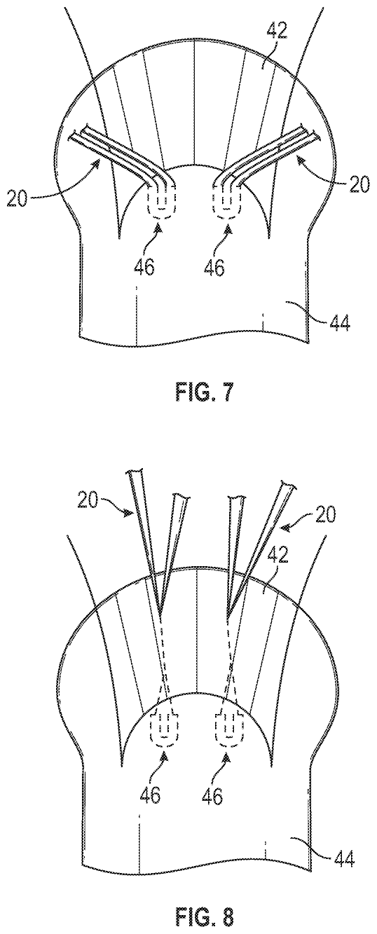

FIGS. 1-6 illustrate an exemplary locking ferrule 10 that can be used when performing various tensionable knotless tissue repairs. For example, the locking ferrule 10 could be utilized during surgical methods for attaching tissue (e.g., ligament, tendon, graft, etc.) to bone or for repairing any other type of tissue effect. The locking ferrule 10 could be used in conjunction with a variety of orthopedic surgical repairs, including but not limited to rotator cuff repairs, Achilles tendon repairs, patellar tendon repairs, and biceps tendon repairs, among many others.

The locking ferrule 10 may include a body 12 that extends along a longitudinal axis A between a proximal end 14 and a distal end 16. The body 12 may be tubular shaped and may be constructed from either metallic materials or plastic materials. However, the specific size, shape, and material make-up of the body 12 are not intended to limit this disclosure.

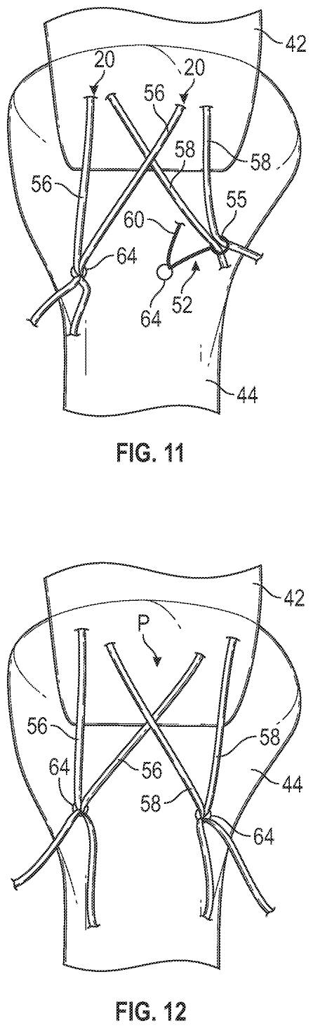

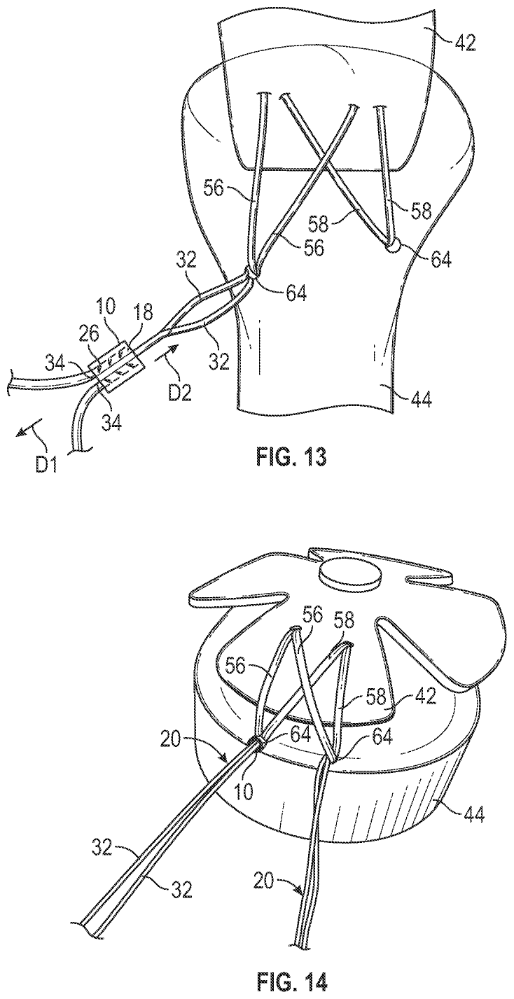

A cannulation 18 may extend through the body 12 and may establish an internal passageway for accommodating one or more strands of suture 20 (see, for example, FIGS. 5 and 6). The cannulation 18 may extend across an entire length of the body 12 and thus extends from the proximal end 14 to the distal end 16. The longitudinal axis A may bisect the cannulation 18.

The body 12 may include an outer diameter wall 22 and an inner diameter wall 24. The outer diameter wall 22 may be smooth or could alternatively include threads, barbs, or other features for facilitating bone fixation. The inner diameter wall 24 may circumscribe the cannulation 18. In some embodiments, the cannulation 18 may taper in a direction toward the distal end 16 and is therefore narrower within the distal end 16 compared to within the proximal end 14.

A plurality of locking barbs 26 may protrude inwardly from the inner diameter wall 24. The locking barbs 26 may therefore occupy at least a portion of the open space of the cannulation 18. In an embodiment, the locking barbs 26 are integrally formed (e.g., molded) features of the body 12 of the locking ferrule 10. The locking barbs 26 may be provided along an entire length of the cannulation 18 or at only select portions thereof. The locking barbs 26 may be either rigid or flexible structures.

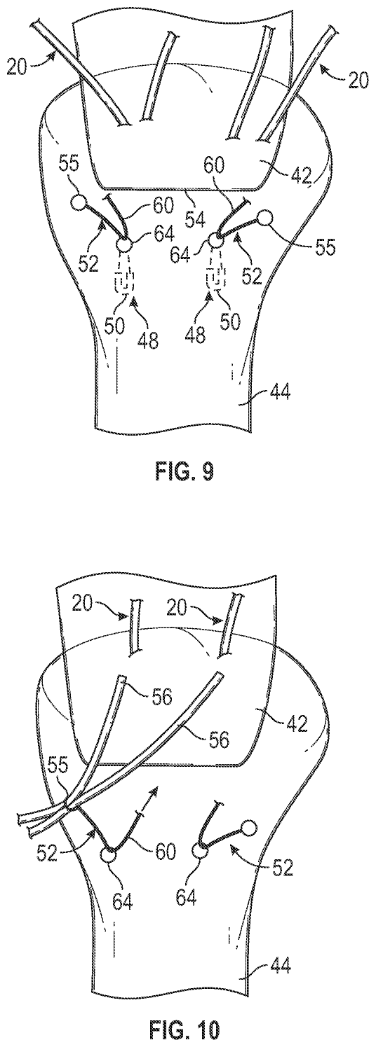

The locking barbs 26 may be arranged in multiple rows along the length of the cannulation 18. For example, the locking barbs 26 may be arranged in a least a first row R1 and a second row R2 (see FIG. 4). In an embodiment, the locking barbs 26 of the second row R2 are staggered relative to the locking barbs 26 of the first row R1 (see FIG. 4).

Each locking barb 26 may include a sharp or pointed tip 28, and each locking barb 26 may be angled in a direction toward the proximal end 14. The locking barbs 26 may therefore establish a one-way locking mechanism that permits one or more sutures 20 to pass through the cannulation in a first direction D1 while preventing the suture(s) 20 from being tensioned or otherwise moved in a second direction D2. The staggered relationship of the rows of locking barbs 26 may provide for maximum engagement with the suture 20 one it is passed through the cannulation 18.

Referring now primarily to FIGS. 5 and 6, with continued reference to FIGS. 1-4, one or more sutures 20 may be passed through the cannulation 18. The suture 20 may be FiberWire®, FiberTape®, or any other suitable suture product. FiberWire® and FiberTape® are suture products marketed and sold by Arthrex, Inc. However, other suture products could be utilized for the suture 20 within the scope of this disclosure. The size and type of suture utilized in conjunction with the locking ferrule 10 are not intended to limit this disclosure.

The suture 20 may include a varying thickness. The suture 20 may therefore include one or more tapered regions 30 where the suture 20 transitions between a thickened section 32 and a thinned section 34. The thinned sections 34 can facilitate passing the suture 20 through the cannulation 18, such as via a suitable suture loader 36 that can be provided as part of a suture locking system that includes the locking ferrule 10, for example, and the thickened sections 32 can provide greater surface area for the locking barbs 26 to engage in order to sufficiently lock the suture 20 relative to the locking ferrule 10.

In an embodiment, the thickened sections 32 of the suture 20 are about twice as thick as the thinned sections 34. However, other ratios (e.g., 1.5:1, 3:1, etc.) between the relative thicknesses (e.g., outer diameters) of the thickened section 32 and the thinned section 34 are contemplated within the scope of this disclosure.

The one or more sutures 20 may be passed through the cannulation 18 of the locking ferrule 10 using the suture loader 36. An eyelet 38 of the suture loader 36 may be passed through the cannulation 18 (e.g., by inserting the eyelet 38 at the distal end 16 of the body 12 and then moving the suture loader 36 in the second direction D2). One or more thinned sections 34 of the suture 20 may then be loaded through the eyelet 38. The suture loader 36 may then be pulled, via a handle 40, in the first direction D1 to pass the suture 20 through the cannulation 18.

Once the suture 20 has been passed through the cannulation 18, the suture 20 may be further tensioned in the first direction D1 to lock the suture 20 relative to the locking ferrule 10. The locking barbs 26 prevent the suture 20 from backing up or otherwise moving in the second direction D2. The pointed tips 28 of the locking barbs 26 may interdigitate with one or more of the thickened sections 32 to lock the suture 20 and prevent it from moving in the second direction D2.

Although shown as locking a single folded suture 20 in the above implementations, the locking ferrule 10 could be configured to receive and lock multiple strands of suture and/or sutures of varying sizes.

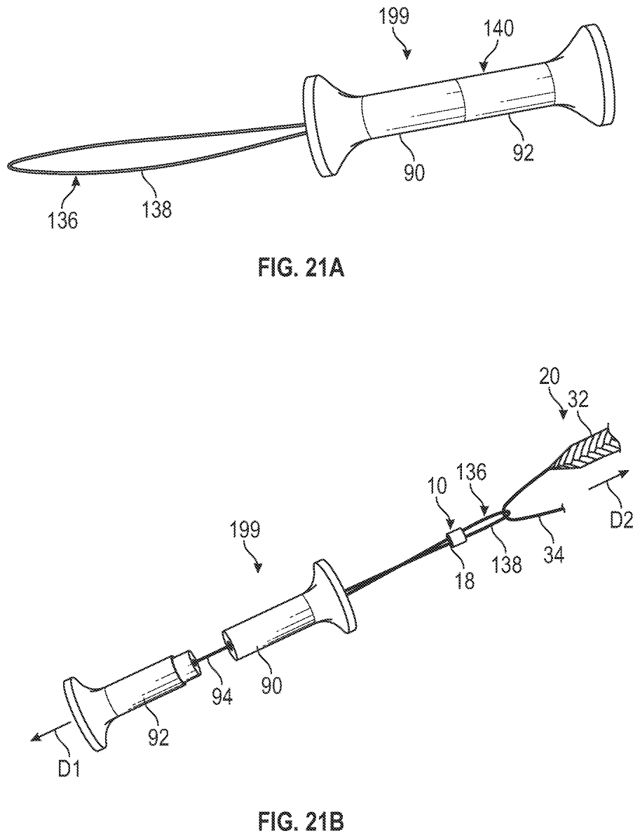

In the implementation described above, a single locking ferrule 10 is utilized to tension and knotlessly fixate the suture 20. However, two or more of the locking ferrules 10 could be utilized together for establish a suture locking system 99 for tensioning and knotlessly fixating the suture 20 (see, e.g., FIG. 20).

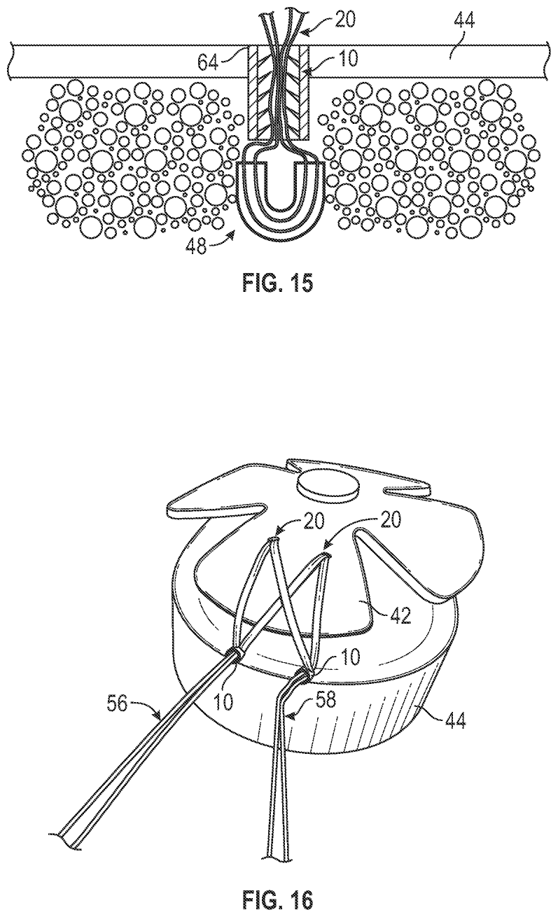

The locking ferrule 10 described above and shown in FIGS. 1-6 may be utilized to tension and knotlessly fixate one or more sutures 20 as part of various surgical methods. FIGS. 7-16 schematically illustrate one such surgical method for attaching a tissue 42 to a bone 44. The tissue 42 may have torn away from the bone 44 during vigorous exercise or sporting activities, for example. When such tears occur, reattachment is often necessary to repair the tissue defect.

The surgical method schematically illustrated in FIGS. 7-16 could be used in conjunction with a variety of orthopedic surgical repairs, including but not limited to rotator cuff repairs, for example. The bone 44 may therefore be associated with any joint of the human musculoskeletal system (e.g., shoulder, knee, hip, ankle, etc.).

In an embodiment, the surgical method is performed as an arthroscopic procedure by working through various arthroscopic portals. However, the exemplary surgical method could alternatively be performed as an open procedure within the scope of this disclosure. The exemplary surgical method may be employed to reduce and then knotlessly attach the tissue 42 to the bone 44 in a manner that enhances footprint compression to maximize tissue-to-bone contact.

Referring first to FIG. 7, after appropriately preparing the bone 44 (e.g., by debriding, creating a bleeding bone bed, preparing bone sockets, etc.), a medial row of fixation devices may be implanted into the bone 44. The medial row of fixation devices may include one or more suture anchors 46. The suture anchors 46 may be knotless suture anchors that do not require the need to tie any knots in the various structures for reducing and securing the tissue 42 to the bone 44 or could be traditional knot tying style suture anchors. In addition, the suture anchors 46 could be “soft” anchors made exclusively of soft, suture-based materials or could be relatively rigid structures made of plastics or metals.

In an embodiment, the suture anchors 46 of the medial row are placed at the articular margin of the bone 44. However, other implantation locations could be selected based on the performing surgeon's preferences. Notably, although two suture anchors 46 are illustrated as being part of the medial row in the illustrated embodiment, a greater or fewer number of suture anchors (or other fixation devices) could be utilized as part of the surgical method within the scope of this disclosure. For example, the medial row could include only a single suture anchor 46.

Each suture anchor 46 may be pre-loaded with one or more sutures 20. The sutures 20 may include individual suture strands, multiple suture strands, suture tape, or any other suture-like product. As shown in FIG. 8, the sutures 20 may be passed upwardly through the tissue 42 after each suture anchor 46 of the medial row is adequately fixated within the bone 44.

Referring now to FIG. 9, the surgical method can proceed by implanting a lateral row of fixation devices into the bone 44. The lateral row of fixation devices may include one or more suture anchors 48. The suture anchors 48 may include an anchor body 50 and a shuttle device 52 received through the anchor body 50. The anchor body 50 may be a “soft” body made exclusively of soft, suture-based materials or could be a relatively rigid plastic or metallic body. The shuttle device 52 may be a passing wire or another suture, for example.

The suture anchors 48 of the lateral row may be placed laterally from an edge 54 of the tissue 42 and slightly distal to the greater tuberosity of the bone 44. The suture anchors 48 may therefore be placed laterally from the suture anchors 46 of the medial row. However, other implantation locations could be selected based on the performing surgeon's preferences and depending on the type of orthopedic procedure being performed. Notably, although two suture anchors 48 are illustrated as being part of the lateral row in the illustrated embodiment, a greater or fewer number of lateral fixation devices could be utilized as part of the surgical method within the scope of this disclosure.

The anchor body 50 of each suture anchor 48 may be inserted into a socket 64 formed in the bone 44. Each socket 64 may be a preformed opening formed in the bone 44 that is sized for receiving the anchor body 50 of one of the suture anchors 48.

The surgical method may next proceed by shuttling the sutures 20 from the suture anchors 46 of the medial row through the anchor bodies 50 of the suture anchors 48 of the lateral row. For example, as shown in FIG. 10, one limb 56 of the suture 20 from each suture anchor 46 may be may be passed through an eyelet 55 of the shuttle device 52 of a first of the suture anchors 48, and then a free end 60 of the shuttle device 52 may be pulled (e.g., in the direction of arrow 62) to allow the limbs 56 to pass through the anchor body 50 of the suture anchor 48. This shuttling process may be repeated to shuttle additional limbs 58 of the sutures 20 from each suture anchor 46 through the anchor body 50 of a second of the suture anchors 48 of the lateral row (see FIG. 11). The limbs 56, 58 may therefore be arranged in a crisscross pattern P that provides a desired area of footprint compression over top of the tissue 42 (see FIG. 12).

At this point of the surgical method, the sutures 20 of the suture anchors 46 are preliminarily fixated relative to the bone 44 by the suture anchors 48. The sutures 20 may subsequently be further tensioned and locked in place using the locking ferrule 10. For example, as shown in FIGS. 13 and 14, after loading the thinned sections 34 of the limbs 56 that are connected to the first of the suture anchors 48 through the cannulation 18 of the locking ferrule 10 (e.g., in the manner shown in FIGS. 5-6), the limbs 56 may be tensioned in the direction D1 to allow the locking ferrule 10 to slide down the limbs 56 in the direction D2 and be received within the socket 64. Further tension of the limbs 56 in the direction D1 allows the pointed tips 28 of the locking barbs 26 to interdigitate with the thickened sections 32 of the sutures 20 to lock the sutures 20 and prevent them from sliding in the second direction D2. Once positioned in the socket 64, the locking ferrule 10 may be positioned over top of the first of the suture anchors 48 of the lateral row (see FIG. 15).

The process shown in FIGS. 13-15 may be repeated to tension and lock the limbs 58 associated with the second of the suture anchors 48 with an additional locking ferrule 10. The final construct achieved by the surgical method is shown in FIG. 16. The excess length of the sutures 20 that extend outside of the cannulation 18 of the locking ferrules 10 may be removed (e.g., cut) once tensioning and locking is complete.

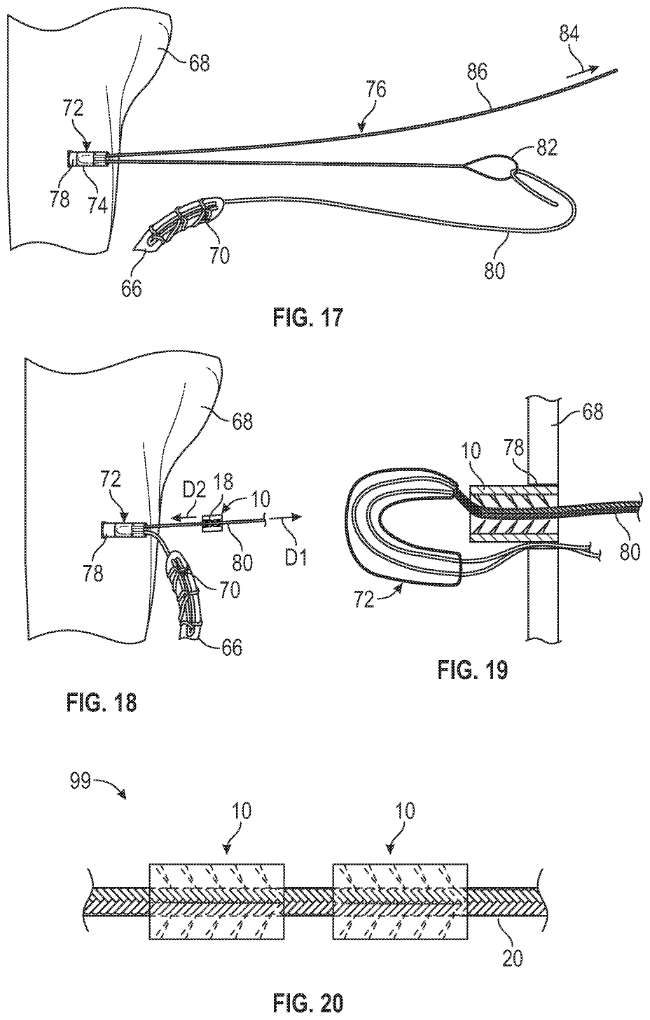

FIGS. 17-19 schematically illustrate another surgical method in which the locking ferrule 10 of FIGS. 1-6 may be utilized to tension and lock suture during a surgical repair. In this embodiment, the surgical method may involve securing a tissue 66 (e.g., a tendon) to a bone 68, such as part of a subpectoral biceps tenodesis procedure, for example.

Referring first to FIG. 17, the surgical method may include creating a looping stitch 70 (e.g., with suture) within the tissue 66 and inserting a suture button 72 within the bone 68 at a desired attachment point for the tissue 66. The suture button 72 may include a button body 74 in the form of a sheath and a shuttle device 76 received through the button body 74. The shuttle device 76 may be a passing wire or a suture, for example.

The button body 74 of the suture button 72 may be inserted into a socket 78 formed in the bone 68. The socket 78 may be a preformed opening formed in the bone 68 that is sized for receiving the button body 74 of the suture button 72.

The surgical method may next proceed by shuttling a suture limb 80 of the looping stitch 70 through the button body 74 of the suture button 72. For example, the suture limb 80 may be passed through an eyelet 82 of the shuttle device 76 of the suture button 72. A free end 86 of the shuttle device 76 may then be pulled (in the direction of arrow 84) to allow the suture limb 80 to pass through the button body 74 of the suture button 72.

At this point of the surgical method, the tissue 66 is reduced into place relative to the bone 68. The suture limb 80 may subsequently be tensioned and locked in place using the locking ferrule 10. For example, as shown in FIG. 18, after loading the suture limb 80 through the cannulation 18 of the locking ferrule 10, the suture limb 80 may be tensioned in the direction D1 to allow the locking ferrule 10 to slide in the direction D2 down and into the socket 78. Further tension of the suture limb 80 allows the pointed tips 28 of the locking barbs 26 to interdigitate with the section of the suture limb 80 accommodated within the cannulation 18, thereby locking the suture limb 80 in place and preventing it from sliding back in the second direction D2. The tissue 66 is therefore securely tensioned and fixated relative to the bone 68. Once positioned in the socket 78, the locking ferrule 10 may be positioned over top of the suture button 72 (see FIG. 19).

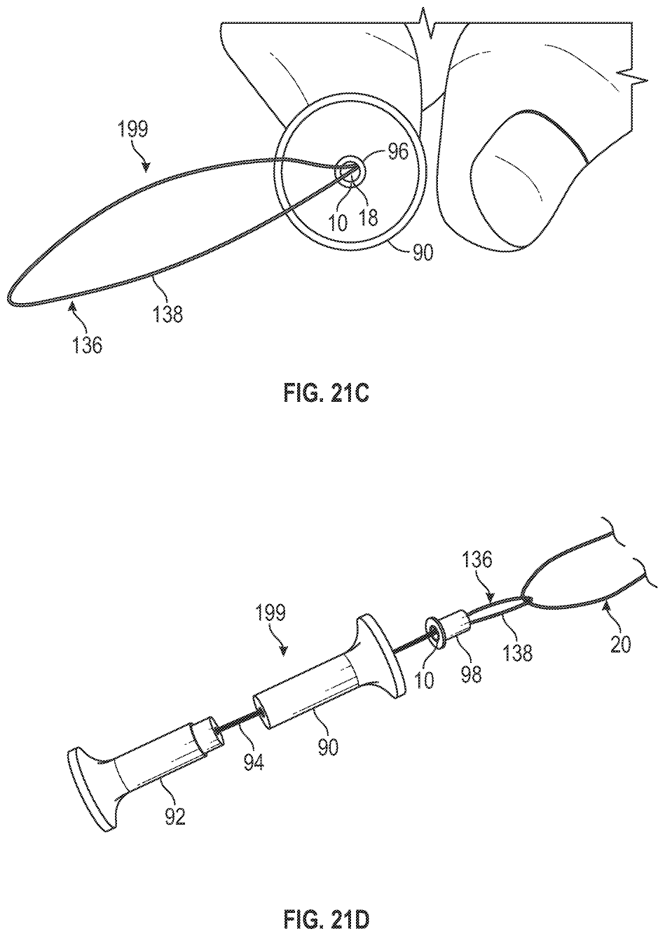

FIGS. 21A-21D illustrate an exemplary suture locking system 199. The suture locking system 199 may include one or more locking ferrules 10 and a suture loader 136 for shuttling a suture 20 through the locking ferrule 10. The locking ferrule 10 may include the design shown in FIGS. 1-4, for example.

The suture loader 136 may include a handle 140 and an eyelet 138. The handle 140 may include a first handle section 90 and a second handle section 92 that is disengageable from the first handle section 90. A proximal section 94 of the eyelet 138 may pass through the first handle section 90 and may be attached to the second handle section 92 such that movement of the second handle section 92 results in moving the eyelet 138. The eyelet 138 may be received through the cannulation 18 of the locking ferrule 10 prior to shuttling the suture 20 therethrough. In an embodiment, the locking ferrule 10 and the suture loader 136 are preassembled to one another prior to performing the suture shuttling procedure.

The thinned section 34 of the suture 20 may be loaded through the eyelet 138 (see FIG. 21B). The second handle section 92 may then be disconnected from the first handle section 90 and then moved in the first direction D1 to shuttle the suture 20 through the cannulation 18 of the locking ferrule 10. Once the suture 20 has been passed through the cannulation 18 of the locking ferrule 10, the suture 20 may be tensioned in the first direction D1 to lock the suture 20 relative to the locking ferrule 10 and prevent movement in the second direction D2 in the manner described above.

In an embodiment, the locking ferrule 10 may be received within a recessed opening 96 of the first handle section 90 of the suture loader 136 prior to shuttling the suture 20 through the locking ferrule 10 (see FIG. 21C). In another embodiment, the locking ferrule 10 may be preloaded within a separate surgical device 98 (e.g., a sleeve, a button, a suture anchor, a screw, a bone plate, an arthroplasty implant, etc.) of the suture locking system 199 prior to shuttling the suture 20 through the locking ferrule 10 (see FIG. 21D).

The locking ferrules of this disclosure may be utilized with one or more additional fixation devices (e.g., anchors, buttons, etc.) for performing various tensionable knotless tissue repairs. The locking ferrules provide for tensioning and retensioning suture(s) at various points of the tissue repair, including subsequent to implantation of the accompanying fixation devices, thus providing numerous advantages over prior tissue repair techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A locking ferrule for performing tissue repairs, comprising:
a body including an outer diameter wall, an inner diameter wall, and a cannulation that is circumscribed by the inner diameter wall; and
a plurality of locking barbs extending into the cannulation and configured to lock a suture that is received through the cannulation relative to the body.

2. The locking ferrule as recited in claim 1, wherein the plurality of locking barbs are integral features of the body.

3. The locking ferrule as recited in claim 1, wherein each of the plurality of locking barbs includes a pointed tip.

4. The locking ferrule as recited in claim 1, wherein the body extends along a longitudinal axis between a proximal end and a distal end.

5. The locking ferrule as recited in claim 4, each of the plurality of locking barbs is angled in a direction toward the proximal end.

6. The locking ferrule as recited in claim 1, wherein the plurality of locking barbs each protrude inwardly from the inner diameter wall.

7. The locking ferrule as recited in claim 6, wherein the plurality of locking barbs are arranged in at least a first row and a second row.

8. The locking ferrule as recited in claim 7, wherein a first portion of the plurality of locking barbs of the first row are staggered relative to a second portion of the plurality of locking barbs of the second row.

9. The locking ferrule as recited in claim 6, wherein the plurality of locking barbs are arranged across an entire length of the body.

10. The locking ferrule as recited in claim 1, wherein the suture includes a varying thickness.

11. A surgical method, comprising:
loading a suture through a cannulation of a locking ferrule;
tensioning the suture in a first direction; and
locking the suture within the cannulation to prevent movement of the suture in a second direction.

12. The surgical method as recited in claim 11, wherein a locking barb of the locking ferrule locks the suture relative to a body of the locking ferrule.

13. The surgical method as recited in claim 12, wherein the locking barb protrudes inwardly from an inner diameter wall of the locking ferrule.

14. The surgical method as recited in claim 12, wherein the locking barb engages a thickened section of the suture to prevent its movement in the second direction.

15. The surgical method as recited in claim 11, wherein loading the suture through the cannulation includes:
pulling the suture through the cannulation in the first direction with a suture loader.

16. The surgical method as recited in claim 15, comprising:
moving an eyelet of the suture loader in the second direction through the cannulation prior to pulling the suture through the cannulation.

17. The surgical method as recited in claim 15, wherein pulling the suture through the cannulation includes:
pulling a thinned section of the suture through the cannulation.

18. A surgical method, comprising:
loading a suture through a cannulation of a locking ferrule;
inserting the locking ferrule into a socket formed in a bone;
tensioning the suture in a first direction relative to the bone; and
locking the suture within the cannulation to prevent movement of the suture in a second direction relative to the bone.

19. The surgical method as recited in claim 18, wherein the locking ferrule is received over top of a fixation device that is positioned within the socket.

20. The surgical method as recited in claim 19, wherein the fixation device is a suture anchor or a suture button.
